(19) United States
(12) Patent Application Publication
(10) Pub. No.: US 2020/0176126 A1
(43) Pub. Date: Jun. 4, 2020

(54) PEER-TO-PEER RATINGS OF MEDICAL EFFECTS

(71) Applicant: Metabyte, Inc., Fremont, CA (US)

(72) Inventors: Manu Mehta, Fremont, CA (US); Lynn Randolph Slater, JR., Pleasanton, CA (US); Nitin Mehta, Fremont, CA (US)

(21) Appl. No.: 16/702,578

(22) Filed: Dec. 4, 2019

Related U.S. Application Data
(60) Provisional application No. 62/774,887, filed on Dec. 4, 2018.

(51) Int. Cl. G16H 70/00; G16H 15/00; G06F 16/21; G06F 16/22; G06F 16/242; G06F 16/2457; G06F 16/25

(52) U.S. Cl. CPC G16H 70/00; G16H 15/00; G06F 16/212; G06Q 30/0208; G06F 16/2445; G06F 16/24573; G06F 16/252; G06F 16/2291

(57) ABSTRACT

A method and a medical rating system (MRS) for providing rating information related to medical items including, for example, medical products, medical services and their effects in a peer-to-peer networking environment while maintaining confidentiality of user information are provided. The MRS receives rating information related to one or more medical items in a peer-to-peer networking environment from multiple user devices. The MRS stores the received rating information and the user information associated with the rating information in separate databases to maintain confidentiality of the user information. The MRS assigns a credibility score to the stored peer-to-peer rating information based on credibility of a rating user and credibility of the stored rating information for the medical items. The MRS renders customized views of the stored rating information for the efficacy and effects associated with each medical item on a graphical user interface of the user device.

### CROSS-REFERENCE TO RELATED APPLICATIONS

This application claims priority to and the benefit of the provisional patent application titled “Peer-To-Peer Ratings Of Medical Effects”, application No. 62/774,887, filed in the United States Patent and Trademark Office on Dec. 4, 2018. The specification of the above referenced patent application is incorporated herein by reference in its entirety.

### BACKGROUND

Medical consumers and medical care providers typically need to know the efficacy and effects of medical products and/or services comprising, for example, medications, medical treatments, medical devices, medical equipment, diagnostic tests, etc., for human or animal use, where the efficacy of a drug product is the degree to which the medical product and/or service achieves its desired effect.

Most conventional methods involving measuring and tracking efficacy and effects of medical products and/or services are based on analyzing a relatively small set of patients during clinical trials prior to receiving regulatory approval for a particular medical product or service. Not only is the sample patient data obtained from a small set of patients during the clinical trials limited, but also testing is generally performed to ensure minimum efficacy requirements and safety criteria are met. Moreover, after regulatory approval, for example, Food and Drug Administration (FDA) approval, when the patient population that uses the medical products and/or services becomes substantially large, the number of people reporting the efficacy and adverse effects of the medical products and/or services remains small relative to the size of the population using the medical products and/or services. Some centralized government organizations, for example, MedWatch in the United States of America, which is a part of the FDA's safety information and adverse event reporting program, provide platforms that allow patients or care providers for humans or animals to report adverse effects or failures. However, such reporting typically happens only when the adverse effects or failure rise to a serious level. Moreover, the flow of information is slow through these platforms due to the information being channeled through centralized government organizations, manufacturers, research institutions, or regulatory organizations. Furthermore, moderate level effects, cross-reactivity, and post-clinical trial efficacy data are often not reported or captured by these platforms. Furthermore, there is minimal reporting or tracking of effects of other medications used by the patient, for example, nature cures, alternate medicines, traditional medicines, home remedies, etc., that are unregulated by the government.

Confidentiality is a significant aspect of reporting effects and side effects of medical products and/or services. Many people would not want their medical history to become public knowledge and will participate in a reporting system only if they are assured of privacy. Furthermore, there are different legal and government policy requirements of privacy in different jurisdictions. Therefore, there is a need for a peer-to-peer rating platform that allows users, for example, patients and care providers for humans or animals to share information about the efficacy, effects, and other usage related information of medical items comprising, for example, medical products, medical services, treatments, tests, etc., on a peer-to-peer basis while maintaining the confidentiality of user information.

Hence, there is a long felt but unresolved need for a method and a medical rating system for providing rating information related to regulated and unregulated medical items and their efficacy and effects to users in a peer-to-peer networking environment while maintaining the confidentiality of user information.

### SUMMARY OF THE INVENTION

This summary is provided to introduce a selection of concepts in a simplified form that are further disclosed in the detailed description of the invention. This summary is not intended to determine the scope of the claimed subject matter.

The method and the peer-to-peer medical rating system disclosed herein address the above recited needs for providing rating information related to regulated and unregulated medical items comprising, for example, medical products, medical services, etc., and their efficacy and effects to users in a peer-to-peer networking environment while maintaining the confidentiality of user information. The peer-to-peer medical rating system allows users, for example, patients and care providers for humans or animals to share information about efficacy, effects, and other usage related information of the medical items on a peer-to-peer basis while maintaining the confidentiality of user information.

The medical rating system comprises a rating information receiving module for receiving rating information related to one or more medical items in a peer-to-peer networking environment from multiple user devices. The rating information comprises a report on efficacy, a report on each response corresponding to a medical item on a numerical value, which may have descriptive text and images. The descriptive text may comprise, for example, visual elements such as images, photographs, or drawings. The medical rating system updates the medical items in a dynamic medical item list. The medical rating system comprises a user information storage module for storing the received rating information and user information in separate databases for maintaining confidentiality of the received user information. For example, the medical rating system stores the received rating information in a ratings database and the user information in a user database. The medical rating system also stores ratings link information in another database, for example, a ratings link database. The medical rating system also comprises a ratings link structure creation module for transforming the stored user information and the rating information into a ratings link table structure. The ratings link structure creation module creates a ratings link table structure comprising the ratings link information for linking the stored rating information to the stored user information. The ratings link table structure comprises predefined fields for storing the ratings link information. The ratings link information is used to connect the rating information stored in the ratings database with the user information stored in the user database. The medical rating system further comprises a credibility score assignment module for assigning a credibility score to the stored rating information based on credibility of a rating user who provides the rating information and credibility of the stored rating information for the medical items. The medical rating system further comprises an analytics module for performing analytics on the dynamic medical item list and the rating information based on predefined criteria. The medical rating system further comprises a customized view module for rendering customized views of the stored rating information for each factor influencing the efficacy and the effects associated with the medical items on a graphical user interface of the user device. The medical rating system also comprises a reward points module for allocating reward points to user accounts of the user for each stored ratings information transmitted from the user devices. The medical rating system also allows users to report moderate level effects, cross-reactivity of drugs, and post-clinical trial efficacy. Furthermore, the medical rating system allows users to report and track effects of unregulated medical items, for example, nature cures, alternate medicine, traditional medicine, home remedies, etc.

### BRIEF DESCRIPTION OF THE DRAWINGS

FIG. 1 illustrates a method for providing rating information related to medical items and their efficacy and effects to users in a peer-to-peer networking environment while maintaining confidentiality of user information.

FIG. 2 exemplarily illustrates a database server of the medical rating system comprising one or more databases for storing user information, rating information, ratings link information, and a list of medical items and effects.

FIG. 3 exemplarily illustrates a block diagram representing the medical rating system architecture.

FIG. 4 exemplarily illustrates a ratings link table structure comprising ratings link information used for linking rating information to user information stored in separate databases.

### DETAILED DESCRIPTION OF THE INVENTION

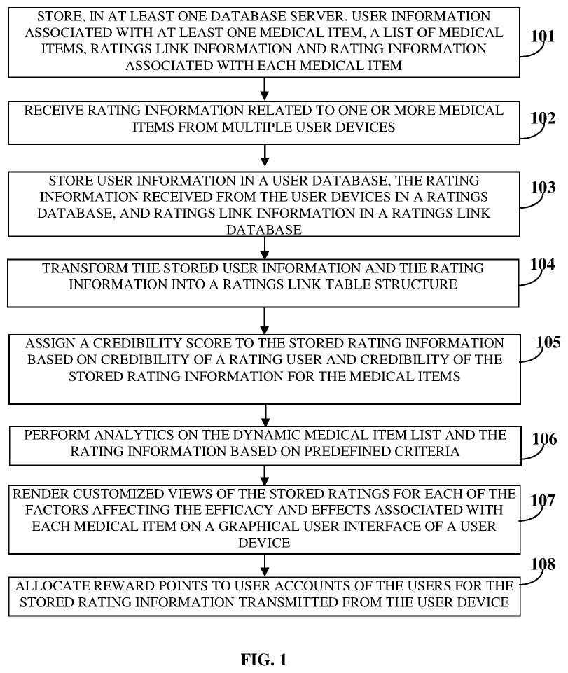

FIG. 1 illustrates a method for providing rating information related to medical items and their efficacy and effects to users in a peer-to-peer networking environment while maintaining confidentiality of user information. As used herein, “medical items” refer to regulated and unregulated medical products and/or medical services comprising, for example, prescription and non-prescription items, medications, medical treatments including conventional medicine such as allopathy, alternate medicines, traditional medicines such as homeopathy, acupuncture, natural medicine, natural cures, home remedies, etc., medical devices, medical tools, medical equipment, diagnostic tests, and any substance intended to have an effect on health such as dietary supplements, infant formulas, user defined concoctions, and medical tools such as a back brace. Also, as used herein, the term “user” refers to any entity or individual, for example, a patient, a patient representative, a care provider, etc., who provides or receives rating information via the medical rating system. A patient refers to any entity or individual, human or animal, who consumes or who is administered a medical item with the expectation of causing an effect on their health. A patient representative refers to any entity or individual that operates on behalf of a patient. For example, the owner of a pet is a patient representative. A care provider refers to any entity such an organization or an individual that recommends or administers medical items. A patient may have many care providers, and a care provider may have many patients. Some medical items may have no care provider, for example, where a patient selects a medication or a medical product that is available without a prescription.

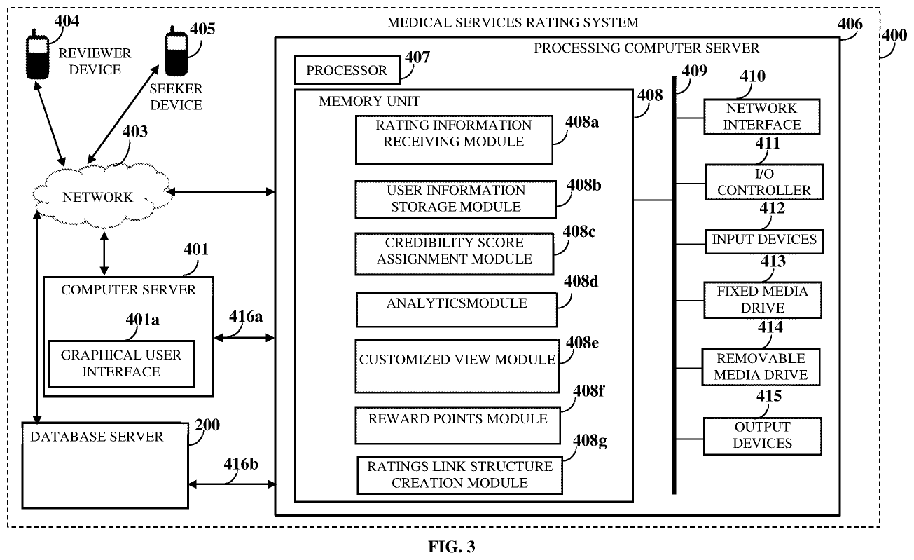

The method disclosed herein employs a medical rating system incorporating a computer system architecture comprising at least one computer server, for example, a processing computer server, a computer server, and a database server, for providing rating information related to one or more medical items and their efficacy and effects to users in a peer-to-peer networking environment while maintaining confidentiality of user information. The medical rating system implements peer-to-peer sharing of ratings and reviews by users, for example, patients and care providers for humans or animals, efficacy, effects, and other use-related information of medical items that are expected to have an effect on the consuming user's health. The computer server of the medical rating system comprises at least one database server and at least one processing computer server. The database server hosts one or more databases for storing user information associated with at least one medical item, a list of medical items herein referred to as a “dynamic medical item list”, ratings link information, and rating information associated with each medical item. The processing computer server comprises at least one processor communicatively coupled to the database server and multiple user devices via a network. As used herein, “user devices” refer to electronic devices, for example, personal computers, laptops, tablet computing devices, smart phones, mobile computers, personal digital assistants, touch centric devices, workstations, client devices, portable electronic devices, network enabled computing devices, interactive network enabled communication devices, etc., possessed by users, for interacting with the medical rating system. The network is, for example, one of internet, an intranet, a wired network, a wireless network, a communication network that implements Bluetooth® of Bluetooth Sig, Inc., a network that implements Wi-Fi® of Wi-Fi Alliance Corporation, a general packet radio service (GPRS) network, a mobile telecommunication network, etc. The processor is configured to execute computer program instructions defined by modules of the medical rating system for rendering rating information related to medical items and their efficacy and effects on at least one graphical user interface (GUI) of a user device associated with a user, while maintaining confidentiality of user information. In an embodiment, the database server and the processing computer server are directly communicatively coupled to the processing computer server as illustrated in FIG. 3.

In the method disclosed herein, the medical rating system, stores 101 in at least one database server, user information associated with at least one medical item, a list of medical items, ratings link information and rating information associated with each medical item. The medical rating system receives rating information related to one or more medical items from multiple user devices in a peer-to-peer networking environment.

The medical rating system allows users to report rating information. Users who provide ratings or rating information via the medical rating system using their user devices are referred to as “rating users”. In an embodiment, the rating information comprises a report on efficacy, a report on each response corresponding to a medical item on a numerical value, which may have descriptive text and images. The descriptive text may comprise, for example, visual elements such as images, photographs, or drawings. An example of the rating information is:

“Aloa verde fixed the rash: 0.7 (report on efficacy), caused headache: 0.2, caused vomiting: 0.1, caused constipation: 0.4 (report on the response)”

The rating information as illustrated above has a numerical rating of 0.7 assigned for efficacy “Aloa verde fixed the rash”. In a similar manner, a numerical rating of 0.2, 0.1, and 0.4, respectively is assigned for each of the response such as headache, vomiting and constipation associated with the usage of Aloa verde.

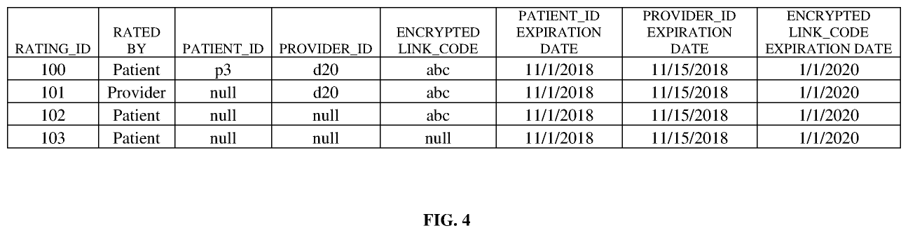

The ratings provide a report on the efficacy, that is, how well the desired result was achieved and on each response or effect. Upon receiving the rating information, the medical items are updated in a dynamic medical item list stored in a medical item database of the medical rating system. The rating information comprises, for example, information about effects and additional factors such as a degree of adherence to a treatment schedule, food and beverages consumed, usage error due to reasons such as forgetfulness, duplicate dosage, poor communications or ambiguous instructions, consumption of other drugs or dietary supplements such as natural vitamins, storage errors such as lack of or improper refrigeration, cost, odor, appearance, taste, texture, time elapsed, etc. The method further comprises storing 103 user information in a user database, the rating information received from each of the user devices in a ratings database, and a ratings link information in a ratings link database separately in the database server. The method further comprises transforming 104 the stored user information and the rating information into a ratings link table structure, exemplarily illustrated in FIG. 4. Transforming the stored user information and the rating information into a ratings link table structure comprises creating a ratings link table structure comprising the ratings link information for linking the stored rating information to the stored user information. The method further comprises assigning 105 a credibility score to the stored ratings information based on credibility of a rating user providing the ratings information, and credibility of the stored ratings information for the medical items. The method further comprises performing 106 analytics on the dynamic medical item list and the ratings information based on a user defined criteria, and a system defined criteria. The method further comprises rendering 107 customized views of the stored ratings information for each of factors and sub-factors affecting the efficacy and the effects associated with each medical item, on a graphical user interface of the user device. The method further comprises allocating 108 reward points to user accounts of the user for each stored ratings information transmitted from the user devices.

The rating information further comprises, for example, patient information, care provider information, and additional data influencing the rating information to allow the rating information to be analyzed without linking back to user information of an identifiable user such as a patient or a care provider, which is stored in a user database of the medical rating system. The patient information comprises patient factors, for example, age, gender, body weight, general health data, species type data, illnesses, progression of illnesses, geographic data, etc., of a patient. The care provider information comprises care provider factors, for example, specialty, type of practice, location of practice, licenses, etc. Factors such as age and type of practice may be copies of information associated with the patient or the care provider. These factors are the stored factors that allow the rating information to be analyzed without linking the rating information to the user information.

In an embodiment, the medical rating system allows the user to report rating information related to a specialty of a medical professional, a sub-specialty of the medical professional, and a super-sub-specialty of the medical professional. For example, an obstetrics and gynecology doctor may specialize in women's reproductive health, and further may have a sub-specialty in fertility treatment such as IVF and may have a super-sub-specialty also, for example IVF specialists for high risk patients or patients with a history.

In an embodiment, the medical rating system allows the care provider to provide rating information related to the super-sub-specialty of the medical professional across multiple skills and traits, for example, C-section of a high risk diabetic patient.

The medical rating system allows a user to add a medical item to the dynamic medical item list stored in the medical item database, for example, using the graphical user interface (GUI) of a user device, to allow the user and other users to rate the newly added medical item or read information about the newly added medical item. Therefore, the dynamic medical item list organically grows and comprises medical items from a broad spectrum, for example, medical treatments including conventional medicine such as allopathy, alternate or traditional therapies medicine such as homeopathy, acupuncture and natural medicine, etc., user defined treatments such as home remedies, medical devices, medical equipment, diagnostic tests, etc., and any products intended to have an effect on health such as dietary supplements, infant formulas or user defined concoctions. In an embodiment, the medical rating system allows a user to vote on whether a medical item is legitimate or spurious. An example of a spurious medical item is a political rant or a business solicitation. The medical rating system designates some users to have editorial powers to allow those users to decide without consideration of additional votes if a medical item is legitimate or spurious. In an embodiment, if the number of votes received for a medical item being spurious exceeds a predefined threshold and is not offset by a predefined level of votes for being legitimate, the medical rating system deletes the spurious medical item and related data stored in the medical item database. The medical rating system limits the viewability of the newly added medical items to a predefined number of users until the newly added medical item receives a predefined threshold level of votes as being legitimate.

The medical rating system also stores and maintains a list of effects of each medical item in the medical item database. The medical rating system allows a user to add an effect, for example, in layman's language, to the stored list of effects, for example, using a graphical user interface (GUI) of a user device, to allow the user and other users to rate the newly added effect or read information about the effect. Therefore, the list of effects that have been identified organically grows. To minimize duplicity, the medical rating system allows a user to vote on whether one reported effect is synonymous with another reported effect. If the number of votes exceeds a system or user defined threshold, the medical rating system merges data on the synonymous effects. In an embodiment, the medical rating system allows a user to vote on whether an effect is legitimate or spurious. An example of a spurious effect is a political rant or a business solicitation. The medical rating system designates some users to have editorial powers to allow those users to decide without consideration of additional votes if an effect is legitimate or spurious. In an embodiment, if the number of votes received for an effect being spurious exceeds a predefined threshold and is not offset by a predefined level of votes for being legitimate, the medical rating system deletes the spurious effect and related data stored in the medical item database. The medical rating system limits the viewability of the newly added effects to a predefined number of users until the newly added effect receives a predefined threshold level of votes as being legitimate.

In an embodiment, the medical rating system allows users to assign personalized names to medical items, for example, “Biji's sun-soaked coconut oil for minor burns”. If multiple publications of a medical item are merged, in an embodiment, the medical rating system lists multiple personalized names under a title similar to, for example, “Also known as”. This provision of the medical rating system is intended to help users memorialize a user, especially the user who either invented the medical item or passed the medical item onto the user. In another embodiment, the medical rating system allows users to assign personalized names to effects of a medical item as the effects of a medical item may be known by different names in different countries or different languages. For example, a migraine may be called a headache accompanied by vomiting. If multiple names of the same effect are reported, in an embodiment, the medical rating system lists the names under a title similar to, for example, “Also known as”.

In an embodiment, the medical rating system allows a user, for example, a patient or a patient representative to identify a care provider associated with a medical item from the stored dynamic medical item list. The medical rating system allows the patient or the patient representative to fill in a care provider profile through a graphical user interface (GUI) of a user device, if the care provider profile is not available in a list of care providers stored in the user database. The medical rating system then invites the care provider to participate as a user in the rating service implemented by the medical rating system. In another embodiment, the medical rating system allows a care provider to identify a patient or a patient representative associated with a medical item from a list of patients or a list of patient representatives respectively, stored in the user database. The medical rating system allows the care provider to fill in a patient profile or a patient representative profile through a GUI of a user device, if the patient profile or the patient representative profile is not available in the list of patients stored in the user database or if the patient or the patient representative is not known to be associated with the care provider. The medical rating system then invites the patient or the patient representative to participate as a user in the rating service implemented by the medical rating system.

The medical rating system stores information about users, for example, patients, patient representatives, care providers, etc., and ratings. To ensure confidentiality, the medical rating system stores the user information comprising, for example, identifiable information about a patient or a care provider separately from the rating information and related tags and analytical properties, and maintains another storage that, in some cases, links the user information to the rating information, that is, links the users to the ratings. The medical rating system stores the user information in the user database and the rating information received from each of the user devices in a ratings database of the medical rating system separately to maintain confidentiality of the received user information. The medical rating system also stores ratings link information in a ratings link database of the medical rating system as disclosed in the detailed description of FIG. 3. The medical rating system uses the stored ratings link information for linking the rating information stored in the ratings database to the user information stored in the user database. The ratings link information is the only means to link the rating information stored in the ratings link database with the user information stored in the user database. The medical rating system also stores shared lists, for example, lists of medical items and effects. In an embodiment, the medical rating system stores information about the medical item, for example, instructions to make or use the medical item, background, history, cost, and recognition of any organizations or people. This information comprises, for example, text, graphics, images, photographs, videos, and audio recordings.

In an embodiment, the medical rating system separately stores the rating information and the credibility score obtained for the rating information related to the specialty of the medical professional, sub-specialty of the medical professional, and super-sub-specialty of the medical professional.

The medical rating system further comprises a credibility module for assigning a credibility score to the stored rating information based on the credibility of the rating user who provides the rating information, and the credibility of the stored rating information for the medical items. For example, a care provider who has built a history with the medical rating system may have a higher credibility score than a new user. In an embodiment, a user's rating or review is weighed according to the credibility assigned to the rating, review, or the user. As the number of ratings for specific effects of a medical item grows, the credibility of the corresponding rating also increases. In an embodiment, the medical rating system integrates with third party computer systems, for example, electronic medical records (EMRs) to authenticate a user and assign a credibility score to the user.

The medical rating system performs analytics on the dynamic medical item list and the rating information based on predefined criteria. The medical rating system allows analytics of the data by sorting or aggregating the data based on user defined criteria or system defined criteria. For example, the medical rating system determines whether a medical item is being reported as effective for the general population or whether there are any sub-populations wherein the medical item is less effective such as men over 40 years of age. Specifically, the algorithm sorts the ratings based on the age group of the various users. Furthermore, an analysis is performed to check if there is a certain trend or pattern in the ratings based on the age groups. For example, the effectiveness of the medical item may correspond to a numerical rating of 0.9 (on a scale of 0 to 1) for an age group below 40 years and the numerical rating may be 0.3 for an age group greater than 40 years. Such a difference may be compared to a threshold value and if the difference is greater than the threshold value, then a warning signal may be output.

In another example, the medical rating system determines whether a factor such as climate affects the reported effectiveness of a medical item. In an exemplary embodiment, parameters corresponding to climate such as, but not limited to, temperature, humidity, air pollution index may be input for the corresponding users and an analysis is performed to understand the dependence of corresponding ratings on such parameters. Additionally, the system may output a warning signal based on the analysis to the user. Such an analysis would also benefit the manufacturers of the medical items by allowing them to make improvements in order to reduce the dependency. In another example, the medical rating system compares the reported effectiveness of a medical item with the effectiveness as claimed by a manufacturer or a provider of that medical item. In another example, the medical rating system allows a care provider to compare the reported effectiveness and reported responses of different medical items.

The medical rating system allows sections and sub-sections to separately rate and review multiple providers of the same medical item, for example, multiple doctors performing a coronary endoscopy procedure, or multiple manufacturers making the same generic drug. The aggregation of numerical ratings from multiple rating users across each individual effect provides a meaningful, actionable insight in a snapshot to a user who analyzes the rating information as opposed to a myriad of text reviews in conventional systems that need to be individually read and understood. Moreover, instead of rating a treatment or a product as overall good or bad, the medical rating system allows rating of specific effects on a numeric scale to allow a user to find the best fit for a situation. In an embodiment, the medical rating system analyzes the rating information to provide views on each individual factor affecting the efficacy and effect of a medical item. The medical rating system renders customized views of the stored ratings for each of the factors and sub-factors affecting the efficacy and the effects associated with the medical item, on a graphical user interface (GUI) of a user device. In an embodiment, the medical rating system renders system specific views for each of the factors and sub-factors affecting the medical item. In an embodiment, the medical rating system tags the medical items and related information comprising, for example, rating information for facilitating the implementation of analytical features comprising, for example, search, sort, and group creation. The medical rating system allows the rating information to be viewed, analyzed, and/or rolled up in aggregate or separately for different effects, factors or sub-factors.

In an embodiment, the medical rating system allocates reward points to user accounts of the user for each stored rating information transmitted from each of the user devices. The medical rating system implements a rewards program to issue incentives, for example, reward points, tokens, etc., to user accounts of the users for using the medical rating system and providing peer-to-peer rating information. The incentives earned by a user may be later exchanged by the user, for example, for medical items, healthcare and non-healthcare related products and/or services such as vitamins, acupuncture, retail gift cards, vacations, cash, etc.

The method and the medical rating system disclosed herein implement peer-to-peer mass reporting of ratings that allows fast discovery and sharing of rating information related to both regulated and unregulated medical items between users in a peer-to-peer networking environment, while maintaining the confidentiality of the user information. By implementing peer-to-peer mass reporting of ratings, the medical rating system provides improved choices of medical items based on the ratings of other users and shares a user's observation of effects of medical items with other users including patients, patient representatives, care providers, manufacturers, news media, and government organizations with instantaneous speed. Moreover, by implementing peer-to-peer mass reporting of ratings, the medical rating system identifies uncommon effects which may have been missed during clinical trials because the medical rating system receives ratings from a substantially larger number of users than the numbers that likely participated in clinical trials. Through the medical rating system, a user may discover that a user's reaction to a medical item is not that uncommon. Furthermore, by implementing peer-to-peer mass reporting of ratings, the medical rating system identifies long term effects relatively fast and moderate level effects, cross-reactivity of drugs, and post-clinical trial efficacy. The medical rating system provides individual ratings for each of the factors affecting a medical item as opposed to providing overall ratings of a product or a treatment. The medical rating system provides assistance in improving the speed of research and development with which new drugs, treatments, devices, equipment, and tests may be developed or existing ones enhanced. The medical rating system further provides a platform to a substantially larger number of users for sharing data on the effects of unconventional, traditional medicines, nature cures, alternate medicines, and home remedies to allow users to make insightful decisions.

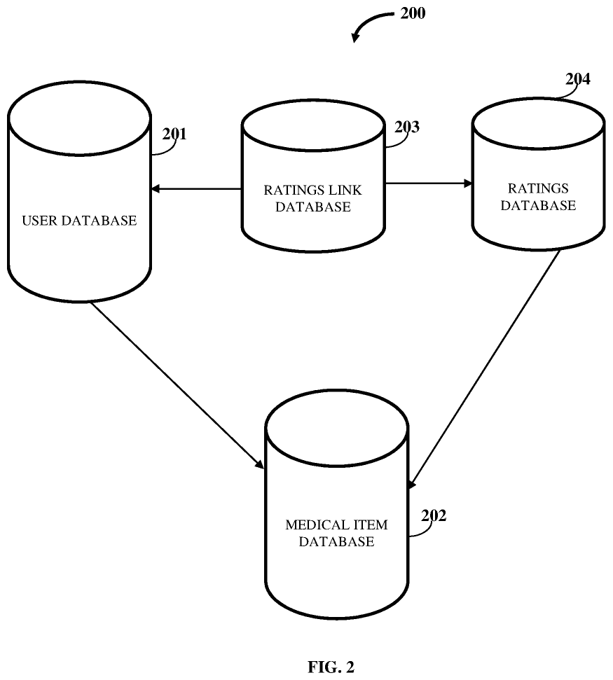

FIG. 2 exemplarily illustrates the database server 200 of the medical rating system comprising one or more databases for storing user information, rating information, ratings link information, and lists of medical items and effects. In an embodiment, storage areas of the medical rating system are provided in a single database, whereas in another embodiment, the storage areas are provided in separate databases on the same or different platforms. The medical rating system stores the user information, the rating information, the ratings link information, and the lists of medical items and effects separately in the database server 200 for maintaining confidentiality of the user information. As exemplarily illustrated in FIG. 2, the database server 200 comprises the user database 201, the medical item database 202, the ratings link database 203, and the ratings database 204. The user database 201 stores user information comprising, for example, patient information, patient representative information, and care provider information associated with at least one medical item as disclosed in the detailed description of FIG. 1. In an embodiment, the medical rating system allows users to enter the user information through graphical user interfaces (GUIs) rendered by the medical rating system on the user devices for storage in the user database 201. In another embodiment, the medical rating system captures the user information from third party computer systems, for example, from electronic medical records (EMRs). In another embodiment, the medical rating system stores the user information received from both user input via the GUIs and the third party computer systems in the user database 201.

The medical item database 202 stores the lists of medical items and effects. The medical item database 202 is a shared or replicated storage database that stores definitions of medical items and effects. The user database 201 and the ratings database 204 share the medical item database 202. The medical item database 202 also stores information about the medical item, for example, instructions to make or use the medical item, background, history, cost, recognition of any organizations or people, etc., in the form of text, graphics, images, photographs, videos, and audio recordings. The ratings link database 203 stores the ratings link information, while the ratings database 204 stores anonymous rating information associated with each medical item and received from multiple user devices in a peer-to-peer networking environment to maintain confidentiality of the user information as disclosed in the detailed description of FIG. 1. The medical rating system uses the stored ratings link information in the ratings link database 203 to connect the anonymous rating information stored in the ratings database 204 with the user information stored in the user database 201.

The medical rating system assures privacy and confidentiality of the user information by not storing any links and thereby disallowing association of ratings with their sources, that is, the rating users. The medical rating system does not provide links from the ratings database 204 containing the rating information to the ratings link database 203 containing the ratings link information, and the user database 201 containing the identifiable user information. Furthermore, the medical rating system does not provide links from the user database 201 containing the identifiable user information to the rating links database 203 and the ratings database 204. The medical rating system only provides an association between the rating users and the ratings via the rating links stored in the ratings link database 203. Despite a user having logged into the medical rating system where a user account comprises a user's identity, the medical rating system does not allow determination of which ratings were provided by that user. Therefore, the only way to access the user information stored in the user database 201 is by accessing the rating link information stored in the ratings link database 203. Furthermore, the rating information comprises user factors such that the rating information can be analyzed without linking the rating information back to the user information.

In an embodiment, the medical rating system is built on one or more centralized servers. In another embodiment, the medical rating system is built on a distributed ledger technology platform such as a blockchain. In another embodiment, the medical rating system uses a combination of techniques where some modules and components of the medical rating system are built on one or more centralized servers and some on the blockchain.

FIG. 3 exemplarily illustrates a block diagram representing the medical rating system architecture 400. The medical rating system 400 comprises at least one computer server 401 for providing rating information related to one or more medical items and their efficacy and effects to users in a peer-to-peer networking environment while maintaining confidentiality of user information. The medical rating system 400 is a computer system that is programmable using a high level computer programming language. In an embodiment, the medical rating system 400 uses programmed and purposeful hardware. In an embodiment, the medical rating system 400 is implemented on a computing device, for example, a personal computer, a tablet computing device, a mobile computer, a portable computing device, a laptop, a touch device, a workstation, a server, portable electronic device, a network enabled computing device, an interactive network enabled communication device, any other suitable computing equipment, combinations of multiple pieces of computing equipment, etc. In an embodiment, the computing equipment is used to implement applications such as media playback applications, a web browser, an electronic mail (email) application, a calendar application, etc. In another embodiment, the computing equipment, for example, one or more servers are associated with one or more online services. In an embodiment, the medical rating system 400 is configured as a web based platform, for example, a website hosted on a server or a network of servers.

The medical rating system 400 comprises at least one computer server 401, at least one database server 200, and at least one processing computer server 406. The computer server 401 renders graphical user interfaces 401a to multiple seeker devices 405 and multiple reviewer devices 404. As used herein, “seeker devices” and “reviewer devices” are user devices, for example, personal computers, laptops, tablet computing devices, mobile computers, mobile phones, smartphones, portable computing devices, personal digital assistants, laptops, wearable computing devices such as the Google Glass® of Google Inc., the Apple Watch® of Apple Inc., etc., touch centric devices, workstations, client devices, portable electronic devices, network enabled computing devices, interactive network enabled communication devices, any other suitable computing equipment, combinations of multiple pieces of computing equipment, etc., possessed by the opportunity seekers and the reviewers respectively, for interacting with the medical rating system 400. In an embodiment, the seeker devices 405 and reviewer devices 404 are hybrid computing devices that combine the functionality of multiple devices. Examples of a hybrid computing device comprise a cellular telephone that includes a media player functionality, a gaming device that includes a wireless communications capability, a cellular telephone that includes a document reader and multimedia functions, and a portable device that has network browsing, document rendering, and network communication capabilities. For purposes of illustration, the seeker devices 405 and reviewer devices 404 are user devices of a recruitment system of entities such as offices, educational institutes, etc.

The processing computer server 406 comprises at least one processor 407. The processing computer server 406 is communicatively coupled to the database server 200 via a network 403. The network 403 is, for example, one of the internet, an intranet, a wired network, a wireless network, a communication network that implements Bluetooth® of Bluetooth Sig, Inc., a network that implements Wi-Fi® of Wi-Fi Alliance Corporation, an ultra-wideband communication network (UWB), a wireless universal serial bus (USB) communication network, a communication network that implements ZigBee® of ZigBee Alliance Corporation, a general packet radio service (GPRS) network, a mobile telecommunication network such as a global system for mobile (GSM) communications network, a code division multiple access (CDMA) network, a third generation (3G) mobile communication network, a fourth generation (4G) mobile communication network, a fifth generation (5G) mobile communication network, a long-term evolution (LTE) mobile communication network, a public telephone network, etc., a local area network, a wide area network, an internet connection network, an infrared communication network, etc., or a network formed from any combination of these networks. In an embodiment, the medical rating system 400 is accessible to the satellite internet of users, for example, through a broad spectrum of technologies and devices such as cellular phones, tablet computing devices, etc., with access to the internet. In an embodiment, the processing computer server 406 and the database server 200 are directly communicatively coupled to the processing computer server through communication links 416a and 416b, as illustrated in FIG. 3.

As exemplarily illustrated in FIG. 3, the medical rating system 400 comprises a non-transitory computer readable storage medium, for example, a memory unit 408 for storing programs and data, and at least one processor 407 communicatively coupled to the non-transitory computer readable storage medium. As used herein, “non-transitory computer readable storage medium” refers to all computer readable media, for example, non-volatile media, volatile media, and transmission media, except for a transitory, propagating signal. Non-volatile media comprise, for example, solid state drives, optical discs or magnetic disks, and other persistent memory volatile media including a dynamic random access memory (DRAM), which typically constitute a main memory. Volatile media comprise, for example, a register memory, a processor cache, a random access memory (RAM), etc. Transmission media comprise, for example, coaxial cables, copper wire, fiber optic cables, modems, etc., including wires that constitute a system bus coupled to the processor 407. The non-transitory computer readable storage medium is configured to store computer program instructions defined by modules, for example, 408a, 408b, 408c, 408d, 408e, 408f, and 408g of the medical rating system 400. The modules 408a, 408b, 408c, 408d, 408e, 408f, and 408g are installed and stored in the memory unit 408 of the medical rating system 100. The memory unit 408 is used for storing program instructions, applications, and data. The memory unit 408 is, for example, a random access memory (RAM) or another type of dynamic storage device that stores information and instructions for execution by the processor 407. The memory unit 408 also stores temporary variables and other intermediate information used during execution of the instructions by the processor 407. The medical rating system 400 further comprises a read only memory (ROM) or another type of static storage device that stores static information and instructions for the processor 407.

The processor 407 is configured to execute the computer program instructions defined by the modules, for example, 408a, 408b, 408c, 408d, 408e, 408f etc., of the medical rating system 400. The processor 407 refers to any of one or more microprocessors, central processing unit (CPU) devices, finite state machines, computers, microcontrollers, digital signal processors, logic, a logic device, an user circuit, an application specific integrated circuit (ASIC), a field-programmable gate array (FPGA), a chip, etc., or any combination thereof, capable of executing computer programs or a series of commands, instructions, or state transitions. In an embodiment, the processor 407 is implemented as a processor set comprising, for example, a programmed microprocessor and a math or graphics co-processor. The processor 407 is selected, for example, from the Intel® processors such as the Itanium® microprocessor or the Pentium® processors, Advanced Micro Devices (AMD®) processors such as the Athlon® processor, UltraSPARC® processors, microSPARC® processors, Hp® processors, International Business Machines (IBM®) processors such as the PowerPC® microprocessor, the MIPS® reduced instruction set computer (RISC) processor of MIPS Technologies, Inc., RISC based computer processors of ARM Holdings, Motorola® processors, Qualcomm® processors, etc. The medical rating system 100 disclosed herein is not limited to employing a processor 407. In an embodiment, the medical rating system 400 employs a controller or a microcontroller.

As exemplarily illustrated in FIG. 1, the medical rating system 400 further comprises a data bus 409, a network interface 410, an input/output (I/O) controller 411, input devices 412, a fixed media drive 413 such as a hard drive, a removable media drive 414 for receiving removable media, output devices 415, etc. The data bus 409 permits communications between the modules, for example, 408a, 408b, 408c, 408d, 408e, 408f, and 408g of the medical rating system 100. The network interface 410 enables connection of the medical rating system 400 to the network 403. In an embodiment, the network interface 410 is provided as an interface card also referred to as a line card. The network interface 410 comprises, for example, one or more of an infrared (IR) interface, an interface implementing Wi-Fi® of Wi-Fi Alliance Corporation, a universal serial bus (USB) interface, a FireWire® interface of Apple Inc., an Ethernet interface, a frame relay interface, a cable interface, a digital subscriber line (DSL) interface, a token ring interface, a peripheral controller interconnect (PCI) interface, a local area network (LAN) interface, a wide area network (WAN) interface, interfaces using serial protocols, interfaces using parallel protocols, Ethernet communication interfaces, asynchronous transfer mode (ATM) interfaces, a high speed serial interface (HSSI), a fiber distributed data interface (FDDI), interfaces based on a transmission control protocol (TCP)/internet protocol (IP), interfaces based on wireless communications technology such as satellite technology, radio frequency (RF) technology, near field communication, etc. The I/O controller 411 controls input actions and output actions performed by the medical rating system 400.

The display screen, via the graphical user interface (GUI) 401a, displays the medical rating. The display screen is, for example, a video display, a liquid crystal display, a plasma display, an organic light emitting diode (OLED) based display, etc. The medical rating system 400 provides the GUI 401a on the display screen. The GUI 401a is, for example, an online web interface, a web based downloadable application interface, a mobile based downloadable application interface, etc. The display screen displays the GUI 401a. The input devices 412 are used for inputting data into the medical rating system 400. The input devices 412 are, for example, a keyboard such as an alphanumeric keyboard, a microphone, a joystick, a pointing device such as a computer mouse, a touch pad, a light pen, a physical button, a touch sensitive display device, a track ball, a pointing stick, any device capable of sensing a tactile input, etc. The output devices 415 output the results of operations performed by the medical rating system 400.

The modules of the medical rating system 400 include a rating information receiving module 408a, a user information storage module 408b, a credibility score assignment module 408c, an analytics module 408d, a customized view module 408e, a rewards point module 408f, and a ratings link structure creation module 408g. These modules have been discussed in detail in FIGS. 1 and 2. The rating information module 408a receives rating information related to one or more medical items from multiple user devices in a peer-to-peer networking environment. The rating information includes a report on efficacy, a report on each response corresponding to the medical item on a numerical value, a descriptive text and images. Upon receiving the rating information, the medical items are updated in a dynamic medical item list stored in a medical item database of the medical rating system 400. The user information storage module 408b stores user information in a user database and the rating information received from each of the multiple user devices 404, and 405 in a ratings database, and a ratings link information in a rating link database separately in the database server 200 to maintain confidentiality of the received user information. The ratings link structure creation module 408g transforms the stored user information and the rating information into a ratings link table structure, exemplarily illustrated in FIG. 4. The ratings link structure creation module 408g creates the ratings link table structure comprising the ratings link information for linking the stored rating information to the stored user information. The ratings link table structure comprises predefined fields for storing the ratings link information comprising at least one of a rating_id field, a ‘rated_by’ field, a ‘patient_id’ field, a ‘provider_id’ field and an encrypted link_code field for each of received information from each of the user devices 404 and 405. The stored (peer-to-peer) ratings link information provides a link to the ratings information stored in the ratings database with the user information stored in the user database. To maintain confidentiality of the received user information, the stored (peer-to-peer) ratings link information is the only stored information to link or connect ratings information stored in the ratings database 204 with the user information stored in the user database 201.

The ratings link information also includes a patient_id expiration date field, a provider_id expiration date field and an encrypted link_code expiration date field. The patient_id field, a provider_id field and an encrypted link_code field is set to null when the current date is past the patient_id expiration date field, a provider_id expiration date field and a link_code expiration date field respectively.

The credibility score assignment module 408c assigns a credibility score to the stored ratings information based on credibility of a rating user providing the ratings information, and credibility of the stored ratings information for the medical items. Furthermore, the analytics module 408d performs analytics on the dynamic medical item list and the ratings information based on a user defined criteria, and a system defined criteria. The customized view module 408e renders customized views of the stored ratings information for each of factors and sub-factors affecting the efficacy and the effects associated with each medical item, on a graphical user interface 401a of the user device. Finally, the reward points module 408f allocates reward points to user accounts of the user for each stored ratings information transmitted from the user devices 404 and 405.

FIG. 4 exemplarily illustrates a ratings link table comprising ratings link information used for linking the rating information to the user information stored in separate databases. The ratings link table of the ratings link database 203 exemplarily illustrated in FIG. 2, comprises ratings link information entered, for example, in a rating_id field, a rated_by field, a patient_id field, a provider_id field, and an encrypted link_code field for each piece of rating information received from each user device. The rating_id field that stores a rating identifier acts as a link identifier. The encrypted link_code is a one-way encrypted link code provided by the rating user in each session and is not stored. Each rating will be present more than once, while some ratings will not be present. The medical rating system uses the rating identifier in the rating_id field of the ratings link table for connecting the rating information stored in the ratings database 204 with the user information stored in the user database 201 exemplarily illustrated in FIG. 2.

The ratings link table further comprises expiration dates entered, for example, in a patient_id expiration date field, a provider_id expiration date field, and an encrypted link_code expiration date field. The medical rating system implements operational settings and user preferences to set the expiration dates. The data value in the patient_id field is set to null when the current date is past the date in the patient_id expiration date field. The data value in the provider_id field is set to null when the current date is past the date in the provider_id expiration date field. The data in the encrypted link_code field is set to null when the current date is past the date in the encrypted link_code expiration date field respectively. The medical rating system deletes the stored peer-to-peer ratings link information in a link record from the ratings link table when the data values in the patient_id field, provider_id field, and the encrypted link code field of the link record are set to null. For example, the link record identified by the rating_id “103” is deleted from the ratings link table as the data values in the patient_id field, the provider_id field, and the encrypted link_code field are set to “null”. The ratings link table exemplarily illustrated in FIG. 3 shows link records in different states of anonymity. The medical rating system maintains anonymity of users, for example, a patient and a care provider by assigning null values to the patient_id field and provider_id field in the ratings link table. In an example, the link record identified by the rating identifier “100” breaks anonymity of the patient identified by the patient_id and the care provider identified by the provider_id only when the encrypted link_code field is active or not set to a null value as exemplarily illustrated in FIG. 3. In another example, the link record identified by the rating identifier “102” breaks anonymity of the patient identified by the patient_id and the care provider identified by the provider_id only when the encrypted link_code field is active or not set to a null value, as both the patient_id field and provider_id field are set to null values as exemplarily illustrated in FIG. 3.

The foregoing examples have been provided merely for explanation and are in no way to be construed as limiting of the method and the medical rating system disclosed herein. While the method and the medical rating system have been described with reference to various embodiments, it is understood that the words, which have been used herein, are words of description and illustration, rather than words of limitation. Furthermore, although the method and the medical rating system have been described herein with reference to particular means, materials, and embodiments, the method and the medical rating system are not intended to be limited to the particulars disclosed herein; rather, the method and the medical rating system extend to all functionally equivalent structures, methods and uses, such as are within the scope of the appended claims. While multiple embodiments are disclosed, it will be understood by those skilled in the art, having the benefit of the teachings of this specification, that the method and the medical rating system disclosed herein are capable of modifications and other embodiments may be effected and changes may be made thereto, without departing from the scope and spirit of the method and the medical rating system disclosed herein.

We claim:

1. A medical rating system incorporating a computer system architecture comprising at least one computer server for providing rating information related to one or more medical items and their efficacy and effects to users in a peer-to-peer networking environment while maintaining confidentiality of user information, the at least one computer server of the medical rating system comprising:
at least one database server hosting one or more databases for storing user information associated with at least one medical item, a list of medical items, ratings link information and rating information associated with each medical item;
at least one processing computer server comprising at least one processor communicatively coupled to the at least one database server and multiple user devices via a network, the at least one processor configured to execute the computer program instructions defined by modules of the medical services rating system comprising:
a rating information receiving module for receiving rating information related to one or more medical items from a plurality of user devices in a peer-to-peer networking environment, wherein each of the rating information comprises a report on efficacy, a report on each response corresponding to the medical item on a numerical value, a descriptive text and images, wherein upon receiving rating information, medical items are updated in a dynamic medical item list stored in a medical item database of the medical rating system;
a user information storage module for storing user information in a user database and the rating information received from each of the user devices in a ratings database and a ratings link information in a rating link database separately in the database server;
a ratings link structure creation module for transforming the stored user information and the rating information into a ratings link table structure, wherein the ratings link structure creation module creates the ratings link table structure comprising the ratings link information for linking the stored rating information to the stored user information, wherein the ratings link table structure comprises predefined fields for storing the ratings link information comprising at least one of a rating_id field, a rated_by field, a patient_id field, a provider_id field and an encrypted link_code field for each of received information from each of the user devices, and wherein the stored ratings link information provides a link to the ratings information stored in the ratings database with the user information stored in the user database;
wherein the ratings link information further comprises a patient_id expiration date field, a provider_id expiration date field and an encrypted link_code expiration date field and a patient_id field, and wherein the provider_id field and the encrypted link_code field are set to null when the current date is past the patient_id expiration date field, a provider_id expiration date field and a link_code expiration date field;
a credibility score assignment module for assigning a credibility score to the stored ratings information based on credibility of a rating user providing the ratings information, and credibility of the stored ratings information for the medical items;
an analytics module for performing analytics on the dynamic medical item list and the ratings information based on a user defined criteria, and a system defined criteria;
a customized view module for rendering customized views of the stored ratings information for each of factors and sub-factors affecting the efficacy and the effects associated with each medical item, on a graphical user interface of the user device; and
a reward points module for allocating reward points to user accounts of the user for each stored ratings information transmitted from the user devices.

2. The medical rating system of claim 1, wherein the system allows the users to add a medical item to the dynamic medical item list stored in the medical item database.

3. The medical rating system of claim 1, wherein the system allows the users to add personalized names to the medical items.

4. The medical rating system of claim 3, wherein the system groups ratings received for each of similar personalized names for each medical service.

5. The medical rating system of claim 1, wherein the system allows the users using the graphical user interface of the user devices to add an effect to the stored list of effect.

6. The medical rating system of claim 5, wherein the system allows the users using the graphical user interface of the user devices to vote on whether an effect is legitimate or spurious.

7. The medical rating system of claim 6, wherein when the number of votes received for an effect being spurious exceeds a predefined threshold the system deletes the spurious effect and related data stored in the medical item database.

8. The medical rating system of claim 6, wherein the system limits a viewability of the newly added effects to a predefined number of users until the newly added effect receives a predefined threshold level of votes as being legitimate.

9. The medical rating system of claim 1, wherein the user is one of a patient and a care provider.

10. The medical rating system of claim 1, wherein the medical item is a regulated medical item, and an unregulated medical item.

11. The medical rating system of claim 10, wherein the regulated medical item and unregulated medical item comprises at least one of a prescription and non-prescription items, medications, medical treatments comprising allopathy, alternate medicine, homeopathy, acupuncture, natural medicine, natural cures, home remedies, medical devices, medical tools, medical equipment, diagnostic tests, and any substance intended to have an effect on health including dietary supplements, infant formulas and user defined concoctions.

12. The medical rating system of claim 1, wherein the system allows the user to report rating information related to a specialty of a medical professional, a sub-specialty of the medical professional, and super-sub-specialty of the medical professional.

13. A method for providing rating information related to one or more medical items and efficacy and effects of the one or more medical items to users in a peer-to-peer networking environment while maintaining confidentiality of user information, the method employing a medical rating system comprising at least one processor configured to execute computer program instructions for performing the method, the method comprising:
storing in at least one database server user information associated with at least one medical item, a list of medical items, ratings link information and rating information associated with each medical item;
receiving rating information related to one or more medical items from a plurality of user devices in a peer-to-peer networking environment, wherein each of the rating information comprises a report on efficacy, a report on each response corresponding to the medical item on a numerical value, a descriptive text and images, wherein upon receiving rating information, medical items are updated in a dynamic medical item list stored in a medical item database of the medical rating system;
storing user information in a user database, the rating information received from each of the user devices in a ratings database, and a ratings link information in a ratings link database separately in the database server;
transforming the stored user information and the rating information into a ratings link table structure, comprising:
creating a ratings link table structure comprising the ratings link information for linking the stored rating information to the stored user information, wherein the ratings link table structure comprises predefined fields for storing the ratings link information comprising at least one of a rating_id field, a rated_by field, a patient_id field, a provider_id field and an encrypted link_code field for each of received information from each of the user devices, wherein the stored ratings link information provides a link to the ratings information stored in the ratings database with the user information stored in the user database;
wherein the ratings link information further comprises a patient_id expiration date field, a provider_id expiration date field and an encrypted link_code expiration date field and a patient_id field, and wherein the provider_id field and the encrypted link_code field are set to null when the current date is past the patient_id expiration date field, a provider_id expiration date field and a link_code expiration date field;
assigning a credibility score to the stored ratings information based on credibility of a rating user providing the ratings information, and credibility of the stored ratings information for the medical items;
performing analytics on the dynamic medical item list and the ratings information based on a user defined criteria, and a system defined criteria;
rendering customized views of the stored ratings information for each of factors and sub-factors affecting the efficacy and the effects associated with each medical item, on a graphical user interface of the user device; and
allocating reward points to user accounts of the user for each stored ratings information transmitted from the user devices.

14. The method of claim 13, further comprising adding a medical item to the dynamic medical list stored in the medical item database.

15. The method of claim 13, further comprising adding personalized names to the medical items.

16. The method of claim 15, further comprising grouping the ratings for each of the similar personalized names.

17. The method of claim 13, further comprising adding an effect to the stored list of effects.

18. The method of claim 17, wherein adding an effect comprises allowing the users to vote on whether an effect is legitimate or spurious.

19. The method of claim 18, wherein allowing the users to vote comprises deleting the spurious effect and related data stored in the medical item database in the event the number of votes received for an effect being spurious exceeds a predefined threshold the system, and wherein adding an effect comprises limiting the viewability of the newly added effects to a predefined number of users until the newly added effect receives a predefined threshold level of votes as being legitimate.

20. A non-transitory computer readable storage medium having embodied thereon, computer program codes comprising instructions executable by at least one processor for providing rating information related to one or more medical items and efficacy and effects of the one or more medical items to users in a peer-to-peer networking environment while maintaining confidentiality of user information, the computer program codes comprising:
a first computer program code for storing in at least one database server user information associated with at least one medical item, a list of medical items, ratings link information and rating information associated with each medical item;
a second computer program code for receiving rating information related to one or more medical items from a plurality of user devices in a peer-to-peer networking environment, wherein each of the rating information comprises a report on efficacy, a report on each response corresponding to the medical item on a numerical value, a descriptive text and images, wherein upon receiving rating information, medical items are updated in a dynamic medical item list stored in a medical item database of the medical rating system;
a third computer program code for storing user information in a user database and the rating information received from each of the user devices in a ratings database and a ratings link information in a rating link database separately in the database server;
a fourth computer program code for transforming the stored user information and the rating information into a ratings link table structure by creating a ratings link table structure comprising the ratings link information for linking the stored rating information to the stored user information, wherein the ratings link table structure comprises predefined fields for storing the ratings link information comprising at least one of a rating_id field, a rated_by field, a patient_id field, a provider_id field and an encrypted link_code field for each of received information from each of the user devices, wherein the stored ratings link information provides a link to the ratings information stored in the ratings database with the user information stored in the user database to maintain confidentiality of the received user information, wherein the ratings link information further comprises a patient_id expiration date field, a provider_id expiration date field and an encrypted link_code expiration date field and a patient_id field, and wherein the provider_id field and the encrypted link_code field are set to null when the current date is past the patient_id expiration date field, a provider_id expiration date field and a link_code expiration date field;
a fifth computer program code for assigning a credibility score to the stored ratings information based on credibility of a rating user providing the ratings information, and credibility of the stored ratings information for the medical items;
a sixth computer program code for performing analytics on the dynamic medical item list and the ratings information based on a user defined criteria, and a system defined criteria;
a seventh computer program code for rendering customized views of the stored ratings information for each of factors and sub-factors affecting the efficacy and the effects associated with each medical item, on a graphical user interface of the user device; and
an eighth computer program code for allocating reward points to user accounts of the user for each stored ratings information transmitted from the user devices.
